(19) United States
(12) Patent Application Publication
(10) Pub. No.: US 2018/0322951 A1
(43) Pub. Date: Nov. 8, 2018

(54) PREDICTION OF ACUTE RESPIRATORY DISEASE SYNDROME (ARDS) BASED ON PATIENTS' PHYSIOLOGICAL RESPONSES

(71) Applicant: KONINKLIJKE PHILIPS N.V., EINDHOVEN (NL)

(72) Inventors: Srinivasan VAIRAVAN, OSSINING, NY (US); Caitlyn Marie CHIOFOLO, NEW HYDE PARK, NY (US); Nicolas Wadih CHBAT, NEW HYDE PARK, NY (US)

(21) Appl. No.: 15/772,135

(22) PCT Filed: Oct. 19, 2016
(86) PCT No.: PCT/IB2016/056264

§ 371 Date: Apr. 30, 2018

Related U.S. Application Data
(60) Provisional application No. 62/249,972, filed on Nov. 3, 2015.

(51) Int. Cl. G16H 50/20; A61B 5/08; G16H 50/30

(52) U.S. Cl. CPC G16H 50/20; G16H 50/50; G16H 50/30; A61B 5/08

(57) ABSTRACT

A process and system for determining a minimal, ‘pruned’ version of the known ARDS model is provided that quantifies the risk of ARDS in terms of physiologic response of the patient, eliminating the more subjective and/or therapeutic features currently used by the conventional ARDS models. This approach provides an accurate tracking of ARDS risk modeled only on the patient's physiological response and observable reactions, and the decision criteria are selected to provide a positive prediction as soon as possible before an onset of ARDS. In addition, the pruning process also allows the ARDS model to be customized for different medical facility sites using selective combinations of risk factors and rules that yield optimized performance. Additionally, predictions may be provided in cases with missing or outdated data by providing estimates of the missing data, and confidence bounds about the predictions based on the variance of the estimates.

### FIELD OF THE INVENTION

This invention relates to the field of computer-aided medical diagnosis, and in particular to an integrated set of models that may be used to predict an onset of ARDS; the parameters of the models being selected for early detection of ARDS.

### BACKGROUND OF THE INVENTION

Acute Respiratory Distress Syndrome (ARDS) is a devastating disease and is characterized by the breakage of the blood-air barrier inducing alveolar flooding and inflammation. ARDS affects over a quarter million patients, causing over four million hospital-days per year. ARDS is estimated to be prevalent in 5-15% of all ICU patients, and the mortality is roughly 40%, and even greater after hospital discharge. Less than one third of ARDS patients are detected by ICU physicians at the bedside. Early detection of ARDS is critical, as it can potentially provide a wider therapeutic window for the prophylaxis and treatment of ARDS and its complications.

An early detection model for ARDS has been disclosed in U.S. patent Ser. No. 14/379,176, “ACUTE LUNG INJURY (ALI)/ACUTE RESPIRATORY DISTRESS SYNDROME (ARDS) ASSESSMENT AND MONITORING”, Vairavan et al., filed 18 Aug. 2014, (hereinafter '176), incorporated by reference herein. The disclosed ARDS detection model provides a continuous score of ARDS risk using knowledge and data based models for detecting ARDS signatures in vitals, lab results, ventilation settings, and so on.

FIG. 1 illustrates an example embodiment of the disclosed ARDS detection system 10. The example input to the model includes:

clinical knowledge sources, including:
clinical knowledge 94 and rules 92 based on the knowledge of medical professions;
clinical research 104 and probabilities 102 based on research articles and other material; and
clinical definitions 114 and logic flow 112 based on existing standards;

pre-ICU (Intensive Care Unit) patient data 144, including demographics, medical history, current condition, and so on; and

ICU patient data 142, including the patient's vital signs, lab results, interventions used, and so on.

In the text and figures of this application, the following abbreviations/acronyms are used. RR—respiratory rate; HR—heart rate; ASBP—arterial systolic blood pressure; ADBP—arterial diastolic blood pressure; Alb—Albumin; Bili—Bilirubin; Hct—Haematocrit; Hgb—Haemoglobin; AS—Aspriration; Pan—Pancreatitis; Pne—Pneumonia; DM—Diabetes Mellitus; Chemo—Chemotherapy; and ADT—Admission Discharge Transfer. The term “APACHE II” is a calculated value based upon AaDO2 or PaO2 (depending upon FiO2), temperature, mean arterial pressure, pH arterial, HR, RR, sodium, potassium, Creatinine, Hct, white blood count, and Glasgow Coma Scale.

A plurality of diagnostic models 90-140 may be used to process the information provided by the input data, each diagnostic model being configured to determine a risk score of a patient's ARDS status, based on the provided information.

FIG. 2 illustrates an example diagnostic model 40 that may be included in the ARDS detection system 10. The diagnostic model 40 uses Lempel-Ziv complexity measures 44, 46 based on a time-series analysis of the patient's physiological data 34, including heart rate (HR), systolic blood pressure (SBP), diastolic blood pressure (DSP), and respiration rate (RR), as well as the treatment 32 that the patient has received thus far. The treatment 32 may include medications 36 or other prescribed interventions, such as invasive ventilation with high tidal volume (VT).

To determine the ARDS status output of the example diagnostic model, a value is computed 50 based on the values of these complexity measures 44, 46. This computed value may be compared to a threshold value 52 (hereinafter ‘thresholding’), and the binary (yes/no) determination is based on the result; for example, if the computed value is greater than or equal to the threshold value, a ‘yes’ is output; otherwise, a ‘no’ is output.

As illustrated in FIG. 1, the ARDS status outputs from the diagnostic models 90-140 are aggregated 82 to provide an estimate of the probability (risk) that the patient will experience ARDS. In an example embodiment, Linear Discriminant Analysis (LDA), or a voting system (SOFALI) may be used to aggregate the predictions from the diagnostic models 90, 100, 110, 120, 130, 140.

If Linear Discrimination Analysis is used to aggregate the outputs of each diagnostic model to determine a probability/likelihood of ARDS, the LDA may receive the analog value that is computed directly 50, rather than the binary output of the threshold function 52.

If a voting system is used, the binary output of each diagnostic model after thresholding 52 may be combined using any of a variety of techniques known in the art, including a weighted or unweighted averaging to determine a probability/likelihood of ARDS.

The probability determined by the aggregator 82 may also be compared to a threshold value to determine whether to issue an alarm or other notification to the medical staff, so that preventive measures or other precautions may be taken.

The binary output of each of the diagnostic models 90, 100, 120, 130, 140, as well as the aggregator 82 (collectively, “the predictors”), may be correct or incorrect, depending upon whether the prediction is retrospectively found to match the actual, or true, outcome (i.e. whether the patient experienced ARDS (‘yes’), or the patient did not experience ARDS (‘no’)). The predictor is said to produce a “false positive” if the predicted outcome is yes, but the actual outcome is no, and is said to produce a “false negative” if the predicted outcome is no, but the actual outcome is yes. Otherwise, the predictor is said to produce a “true positive” (both predicted and actual outcomes are yes), or a “true negative” (both predicted and actual outcomes are no).

A ROC (Receiver Operating Characteristic) curve is commonly used to characterize the ‘quality’ of a predictor, such as illustrated in FIG. 3, wherein the ROC of each of six diagnostic models (A-F) are illustrated, as well as the composite ROC of a SOFALI voting system (G) and an LDA aggregator (H) based on the combination of these six diagnostic models. The ROC curve maps the probability that the predictor will produce a correct positive output (“true positive”) vs. the probability that the predictor will produce an erroneous positive output (“false positive”) for the range of possible threshold values. The proportion of “true positives” of those with the disease is commonly referred to as the “sensitivity” of the predictor, and the proportion of “true negatives” of those without the disease is commonly referred to as the “specificity” of the test; correspondingly, the proportion of “false positives” is equal to 1−specificity.

In a typical predictor, a very high positive threshold value is likely to produce very few false positives, but also fewer true positives than a lower threshold value, corresponding to the lower left region of the ROC space illustrated in FIG. 3. As the threshold value decreases, the number of true positives, as well as the number of false positives, can be expected to occur, corresponding to the upper center region of the ROC space. If the threshold value is very low, the proportion of false positives can be expected to increase, corresponding to the upper right region of the ROC space.

A “useless” predictor is one in which it is equally likely to produce a false positive as it is to produce a true positive, corresponding to the diagonal ROC line 210 of FIG. 2. The predictors that provide the ROC curves A-H produce a larger probability of true positives than false positives, and thus are better predictors than the useless predictor that produced the ROC curve 210. The predictor that provides the ROC curve G, for example, produces a larger probability of true positives and fewer false positives than another predictor that provides the ROC curve C, and thus is a better predictor than this other predictor. The closer a ROC curve is to the upper left corner of the ROC space, the closer the predictor approximates a “perfect” predictor (all true positives and no false positives). In FIG. 3, the predictors that provided the ROC curves H and D are considered to be better predictors than the predictors that provided the ROC curves A-C and E-G.

A statistic used to characterize a predictor's ability to correctly predict the outcome is the area under the ROC curve (AUC, or AUROC). The AUC may range from 0 to 1, and represents the predictor's probability of being able to correctly identify the positive case when presented with a pair of cases in which one case had a positive outcome and the other case had a negative outcome, across the range of thresholds. The AUC is commonly referred to as the “accuracy” of the test.

The choice of the threshold to use when applying the predictor to a case is generally a tradeoff between the likelihood of false positives (“false alarms”) and false negatives (“missed diagnosis”) and the costs or consequences of each of these results. If the costs or consequences of either erroneous prediction is assumed to be the same, the threshold value that produced the point on the knee of the ROC curve is generally selected as the optimal threshold value.

Although the ARDS detection system 10 provides an accuracy (AUC) of nearly 90%, as illustrated by the ROC curve H (AUC: 0.87), this accuracy is achieved by obtaining and assessing a substantial amount of patient information, as illustrated in the above list of abbreviations and acronyms. Although some of this information may be readily available, obtaining other information may require specific tests, some of which may be invasive, or at least uncomfortable. Also, some tests may not be readily available at all medical facilities, or may be infrequently available due to demand, cost, or other factors.

Additionally, the outcome of each predictor at any particular time is based on the available patient information at that time; if a recent value of an input feature is not available, the predictor uses the last available value, and this value may be outdated, resulting in a less accurate, and possibly erroneous, prediction.

If a value is not available for an input feature, the diagnostic model replaces the missing feature with the population median value of that feature. However replacing missing features with their respective population medians is not appropriate for interventional features, such as Tidal Volume or PEEP, as it amounts to falsely inputting an intervention for a patient when the information may be missing because, in fact, no intervention may have been administered.

Further, a number of input features used in the ARDS detection system 10 are somewhat subjective, and other features may be related to therapeutic measures that are taken, although the effectiveness of these measures on the particular patient may be unknown.

### SUMMARY OF THE INVENTION

It would be advantageous to provide an ARDS detection/prediction system that is able to provide a reasonably accurate prediction of ARDS with substantially less patient information than currently required. It would also be advantageous to provide an ARDS detection/prediction system that is able to provide a prediction of ARDS well before the onset of ARDS, so that preventive or protective measures may be taken.

To better address one or more of these concerns, in an embodiment of this invention, a minimal, ‘pruned’ version of the known ARDS model is provided that quantifies the risk of ARDS in terms of physiologic response of the patient, eliminating the more subjective and/or therapeutic features currently used by the conventional ARDS models. This approach provides an accurate tracking of ARDS risk modeled only on the patient's physiological response and observable reactions, and the decision criteria are selected to provide positive predictions as soon as possible before an onset of ARDS. In addition, the pruning process also allows the ARDS model to be customized for different medical facility sites using selective combinations of risk factors and rules that yield optimized performance. Additionally, predictions may be provided in cases with missing or outdated data by providing estimates of the missing data based on prior recorded data.

To provide this optimized ARDS system, each predictor is trained by providing a time series of physiological data of each patient of a plurality of prior patients, an identification of whether the patient experienced ARDS, and a time of ARDS onset for each patient that experienced ARDS. Based on this training, a ROC curve and an area under the ROC curve (AUC) that characterizes the diagnostic model's ability to correctly identify whether a patient will experience ARDS is determined. As contrast to the conventional ARDS predictors, the threshold of the aggregator and the threshold of each diagnostic model, if used to provide an output to the aggregator, may be selected to provide an early prediction of ARDS, based on the recorded prediction times before the onset of ARDS (“prediction lead time”). The selected threshold is stored for the aggregator and each diagnostic model that uses thresholding, to provide early predictions of ARDS in future patients, based on the selected threshold(s).

To compensate for incomplete or obsolete values of required patient data, an artificial value for the missing value is used by the diagnostic model, based on values of the missing feature among a population of prior patients. Because this value is artificial/estimated, a confidence interval about the aggregate likelihood of ARDS is determined and reported.

To further reduce the required input features for the diagnostic models, the sensitivity of each diagnostic model's output to each input feature may be determined, based on the sets of physiological data of prior patients, and the input features having the least impact on the accuracy of the diagnostic model, or the prediction lead-time, may be omitted in a revised version of the diagnostic model. This sensitivity determination may also be taken into account when an input feature is not available at a particular medical facility, and when determining the aforementioned confidence intervals.

Thereafter, the ARDS risk for future patients may be based on this reduced set of required input features and corresponding revised diagnostic models. The threshold(s) for the revised diagnostic model(s) may also be selected to maximize the prediction lead time, or maximize the proportion of patients receiving at least some minimal prediction lead time.

### BRIEF DESCRIPTION OF THE DRAWINGS

The invention is explained in further detail, and by way of example, with reference to the accompanying drawings wherein:

FIG. 1 illustrates an example ARDS detection system as disclosed in the parent application to this application.

FIG. 2 illustrates an example diagnostic model used in the example ARDS detection system of FIG. 1.

FIG. 3 illustrates example ROC curves for six diagnostic models and two prediction aggregators in an example ARDS detection system.

FIG. 4 illustrates an example ARDS detection system of this invention that uses patient physiological data to provide a prediction of ARDS.

FIG. 5 illustrates an example process for determining prediction thresholds that maximize prediction lead times of a plurality of ARDS predictors.

FIG. 6 illustrates an example ‘pruning’ of the ARDS detection system of FIG. 4 to reduce the number of required input features to each diagnostic model.

FIG. 7 illustrates an example flow diagram of a pruning of the ARDS detection system of FIG. 4.

FIG. 8 illustrates a further example pruning of the ARDS detection system of FIG. 6.

FIG. 9 illustrates an example flow diagram for using an embodiment of this invention for detecting an onset of ARDS.

FIG. 10 illustrates example ROC curves corresponding to an example embodiment of the ARDS detection system of this invention.

FIG. 11 illustrates example distribution plots of prediction lead times for different predictor thresholds.

FIG. 12 illustrates an example comparison of ROC curves of the aggregate ARDS between a parsimonious embodiment of this invention and an embodiment of the ARDS detection system of the prior art.

Throughout the drawings, the same reference numerals indicate similar or corresponding features or functions. The drawings are included for illustrative purposes and are not intended to limit the scope of the invention.

### DETAILED DESCRIPTION

In the following description, for purposes of explanation rather than limitation, specific details are set forth such as the particular architecture, interfaces, techniques, etc., in order to provide a thorough understanding of the concepts of the invention. However, it will be apparent to those skilled in the art that the present invention may be practiced in other embodiments, which depart from these specific details. In like manner, the text of this description is directed to the example embodiments as illustrated in the Figures, and is not intended to limit the claimed invention beyond the limits expressly included in the claims. For purposes of simplicity and clarity, detailed descriptions of well-known devices, circuits, and methods are omitted so as not to obscure the description of the present invention with unnecessary detail.

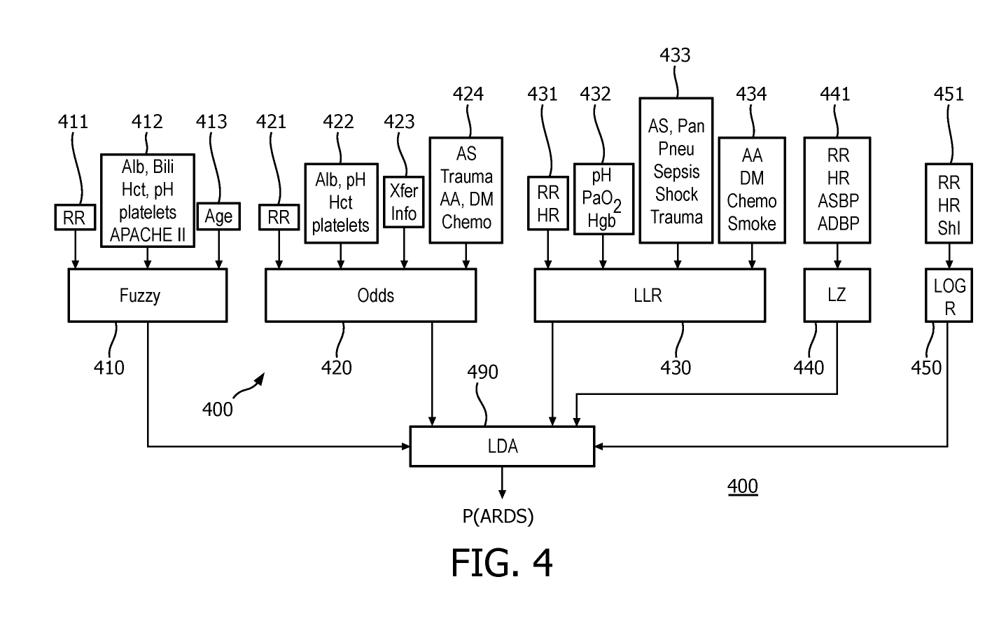

FIG. 4 illustrates an example ARDS detection system 400 that uses patient physiological data to provide a prediction of a future onset of ARDS. The example system 400 includes five diagnostic models 410, 420, 430, 440, and 450. The diagnostic model 410 uses “fuzzy logic” to predict whether or not the current patient will experience ARDS, based on the set of input features 411-413. The diagnostic model 420 uses an “odds ratio” test based on the set of input features 421-424. The diagnostic model 430 uses a “log likelihood ratio” test based on the set of input features 431-434. The diagnostic model 440 uses a “Lempel-Ziv complexity” test based on the set of input features 441; and the diagnostic model 450 uses a “logistic regression” test based on the set of input features 451.

Each of the aforementioned models/tests are well known in the art, as are the techniques used to determine the features associated with each model based on a retrospective analysis of case histories of prior patients. The aforementioned '0176 application, which is incorporated by reference herein, provides a more detailed description of these tests.

Of particular note, only physiological or observable measures are used as input to the example diagnostic models 410, 420, 430, 440, 450. For purposes of this disclosure, the term physiological data, or physiological measure, includes any physical characteristic of a patient, including, for example, age, gender, and so on. Restricting the input to physiological measures reduces the amount of information required, removes subjective information, and provides an ARDS prediction that is independent of the interventions or medications provided, except for their effect on the patient's physiological measures.

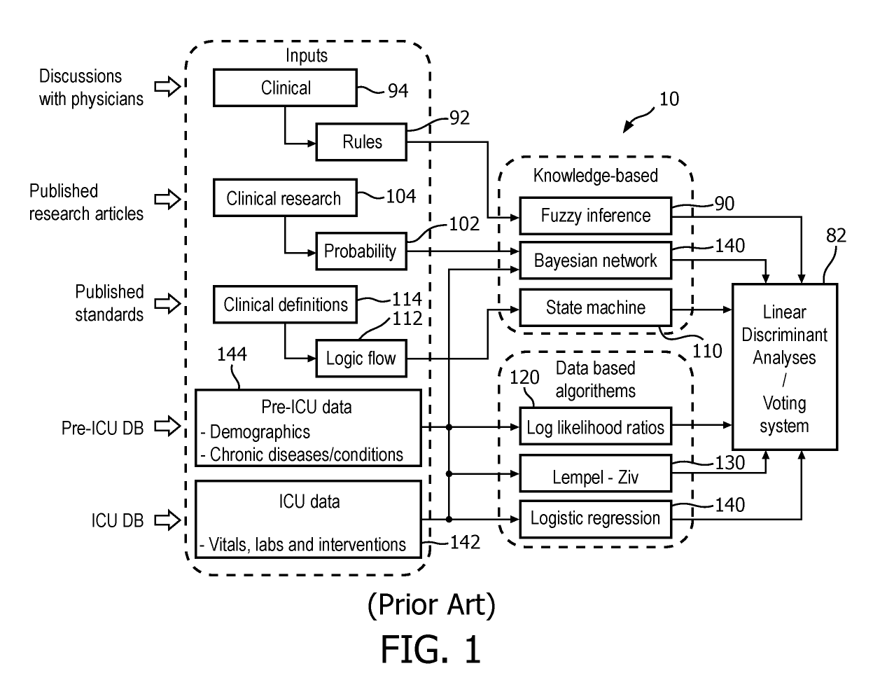

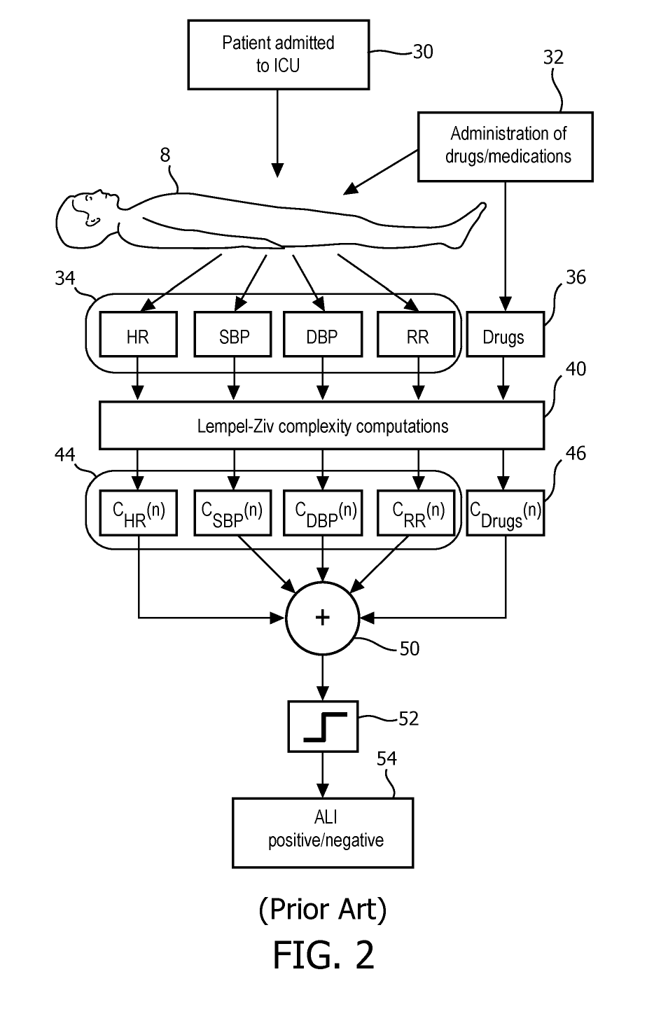

The ARDS status output of each of the diagnostic models 410, 420, 430, 440, and 450 may be determined by comparing a computed value based on the inputs to the diagnostic model to a threshold value associated with each diagnostic model, or by providing a continuous variable if thresholding is not performed, as detailed above with regard to FIGS. 1-3. The ARDS status from the different diagnostic models are aggregated to provide a probability P(ARDS) that the current patient will experience ARDS. In the example embodiment of FIG. 4, a Linear Discrimination Analysis (LDA) is used as the aggregator 490; accordingly, the output of each diagnostic model may be the computed value produced by the diagnostic model, without thresholding. Other aggregation techniques may be used, including for example, a voting technique such as SOFALI, which conventionally uses the binary output of each diagnostic model after thresholding. The aggregator 490 may be configured to provide a binary yes/no prediction of an onset of ARDS, based on a comparison of the determined aggregated value to a threshold value.

Because advanced notice of a positive ARDS prediction has a significant impact on the effectiveness of the prophylaxis and treatment available for ARDS and its complications, the threshold in the aggregator 490 and each of the thresholds used in the diagnostic models 410, 420, 430, 440, 450, if any, are selected to maximize the prediction lead-time before the onset of ARDS.

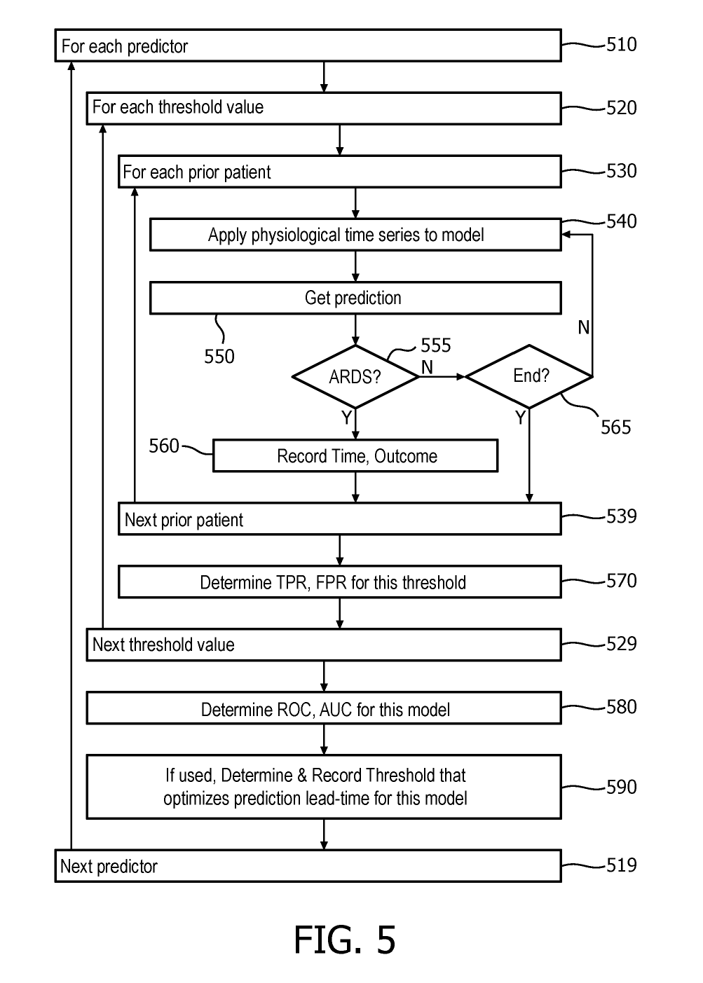

FIG. 5 illustrates an example process for determining prediction thresholds that optimize prediction lead times of a plurality of ARDS diagnostic models 410, 420, 430, 440, 450, and the aggregator 490. For ease of reference, because the diagnostic models and the aggregator provide a prediction of ARDS, the term ‘predictor’ is used herein to refer to diagnostic models 410, 420, 430, 440, 450, and the aggregator 490. In this example embodiment, each predictor is analyzed independently via the loop 510-519.

In the loop 520-529, the performance of the predictor is assessed for each of a plurality of possible threshold values when applied to physiological data associated with prior patients in the loop 530-539. The actual, or true outcome (ARDS, no ARDS) is known for each of these prior patients, as well as the time of the ARDS onset for those patients who experienced ARDS. At 540 the time series of physiological data of each prior patient is applied to the current predictor, and as each new data item is processed by the predictor, a prediction is obtained, at 550. If ARDS is predicted, at 555, the time of this prediction and the actual outcome for this patient, including the time of ARDS onset, is recorded, at 560.

In the example embodiment, the prediction of ARDS onset is maintained as soon as the first positive prediction is provided; that is, subsequent negative predictions do not change this positive prediction. Accordingly, upon receiving a positive ARDS prediction and recording the prediction time and the true outcome (and time), the processing of this prior patient's data is terminated and the next prior patient is selected, at 539.

If a positive ARDS prediction is not reported and the end of the prior patient's data is reached, at 565, the next prior patient is selected, at 539.

After the data of the last prior patient is processed, the loop 530-539 is terminated. At this point, all of the positive predictions using the current threshold value have been recorded, along with the true outcome corresponding to each prediction. For those prior patients who experienced an actual ARDS onset, the difference between the time of the ARDS onset and the time of the positive ARDS prediction provides the prediction lead time for each of these prior patients using the current threshold.

Based on the recorded positive predictions, and actual outcomes, the number of true positives and false positives may be determined, from which the true positive rate TPR and false positive rate FPR may be calculated for the current threshold. This pair of positive rates provide a point on the ROC curve corresponding to this threshold. This pair is recorded, at 570, which facilitates the creation of the ROC curve and corresponding AUC. This pairing of the true positive rate and false positive rate for each threshold also facilitates the selection of a threshold that maximizes the likelihood of having an advanced warning for patients who are determined to be likely to experience ARDS, as detailed further below.

After the set of possible threshold values are processed, at 529, the ROC curve and AUC for this predictor may be determined and presented, at 580.

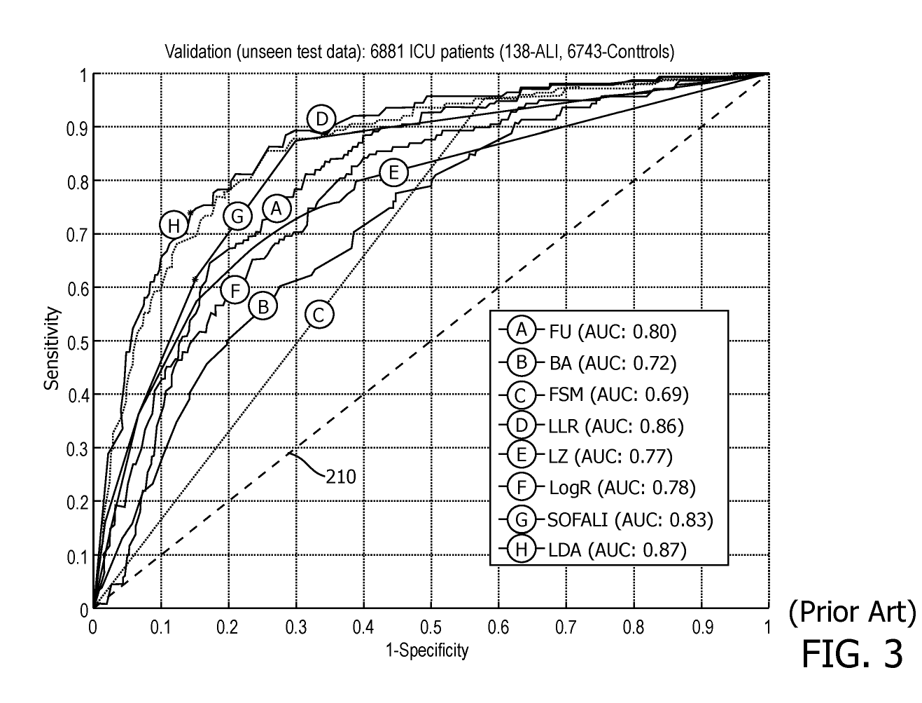

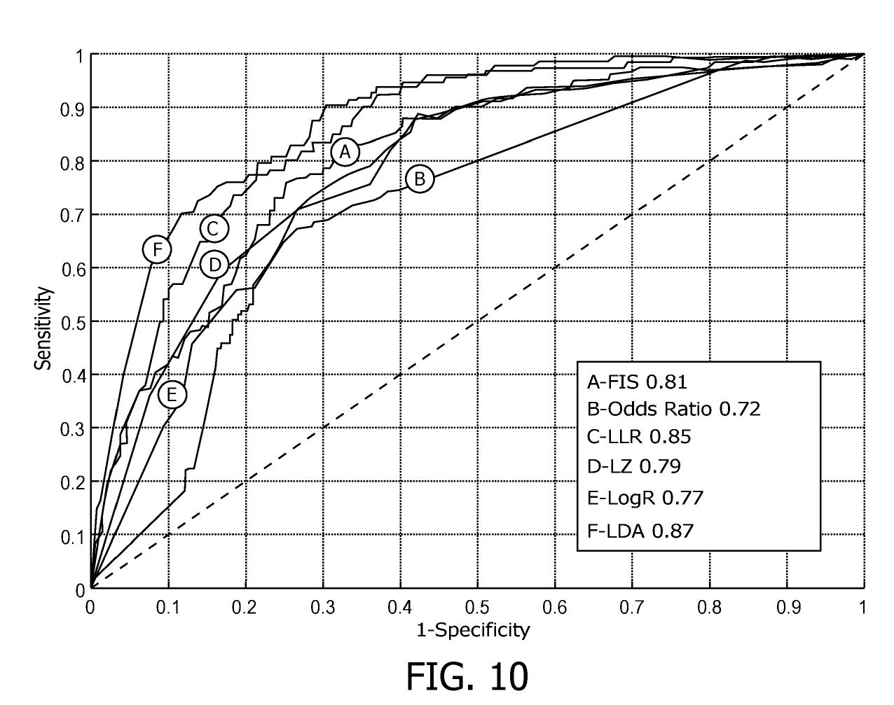

An example set of ROC curves for the five diagnostic models 410, 420, 430, 440, 450 and the aggregator 490 of the ARDS detection system 400 of FIG. 4 is illustrated in FIG. 10. As illustrated in FIG. 10, the aggregator 490 achieves a ROC curve F with an AUC of 0.87, which is comparable to the illustrated ROC curve H of FIG. 3 of the conventional ARDS detection system 10, even though the detection system 400 of FIG. 4 uses substantially fewer input data items, and is not dependent upon interventions or drugs administered during the treatment of the patients.

At 590, a threshold value for the current predictor is selected for use with future patients if the current predictor is the aggregator 490 or a diagnostic model 410, 420, 430, 440, 450 that provides a binary value to the aggregator after thresholding. Using the recorded prediction lead times for each of the true positive predictions using a given threshold (at 560), a threshold may be selected that maximizes the expected prediction lead time for the current predictor. A variety of techniques and criteria may be used to select this threshold. For example, the number/proportion of prior patients that had at least a given minimum lead time may be used as the criteria for selecting the threshold; alternatively, the threshold that provided the longest average lead time may be selected, or the threshold that provided the longest median lead time may be selected, and so on.

In an example embodiment, the cumulative distribution function (cdf) may be used to select the threshold for each test. Example cumulative distribution functions are illustrated in FIG. 11, which shows the distribution of positive predictions with respect to the time of positive prediction before the onset of ARDS (time=0), for each of three threshold values. For example, cdf 1120 shows about 20% (1121) of the positive predictions occurred at least 18 hours before the onset of ARDS, 40% (1122) of the positive predictions occurred at least 1.5 hours before the onset, and about 65% (1123) of the positive predictions occurred at or before the onset of ARDS. The median prediction lead-time was about 4 hours with the threshold that provided cdf 1120. A lower threshold will produce a higher proportion of early detections, as illustrated by cdf 1130; and a higher threshold will produce a lower proportion of early detections, as illustrated by cdf 1110.

Although a lower threshold will provide for a greater proportion of early positive identification of patients who are subsequently found to have experienced ARDS, this lower threshold will also produce a greater proportion of positive identifications of patients who are subsequently found to not have experienced ARDS (false positives). In general, because the consequences of not identifying a patient who is likely to experience ARDS (false negative), is substantially greater than the consequences of mistakenly predicting that a patient is likely to experience ARDS, a relatively large proportion of false alarms (e.g. 20-30%) may be acceptable.

This high rate of false alarms is also acceptable because, during the monitoring of the prior patients, when their physiological condition indicated a likelihood of ARDS, some protective or preventive treatments would have been applied. When the protective or preventive treatments were applied to these patients, the treatments were likely to have been effective in preventing the onset of ARDS for at least a portion of these patients. Although these patients, who would have experienced ARDS had they not received the treatments, were correctly identified by the predictor(s), the fact that the treatments were effective in preventing ARDS caused these patients to be among those considered to have received a “false positive” prediction.

One of skill in the art will recognize that a higher or lower false-alarm proportion may be used, depending upon the nature of the protective or preventive treatments applied to the patients predicted to experience ARDS, and the expected effectiveness of these treatments. In some embodiments, a different proportion of false alarms may be acceptable for each particular predictor, depending upon, for example, the invasiveness of the specific treatment that may be applied if that particular predictor indicates a positive prediction of ARDS.

As noted above, at 570, the true positive rate (TPR) and the false positive rate (FPR) are recorded for each threshold value of each predictor. To provide a maximum proportion of true positive predictions of ARDS, the threshold value that produced a false positive rate equal to the maximum allowable false positive rate is selected.

After all of the predictors are processed to select a threshold that maximizes each predictor's proportion of patients that are correctly identified as likely to experience ARDS, given an allowable false-alarm rate, the process terminates, at 519.

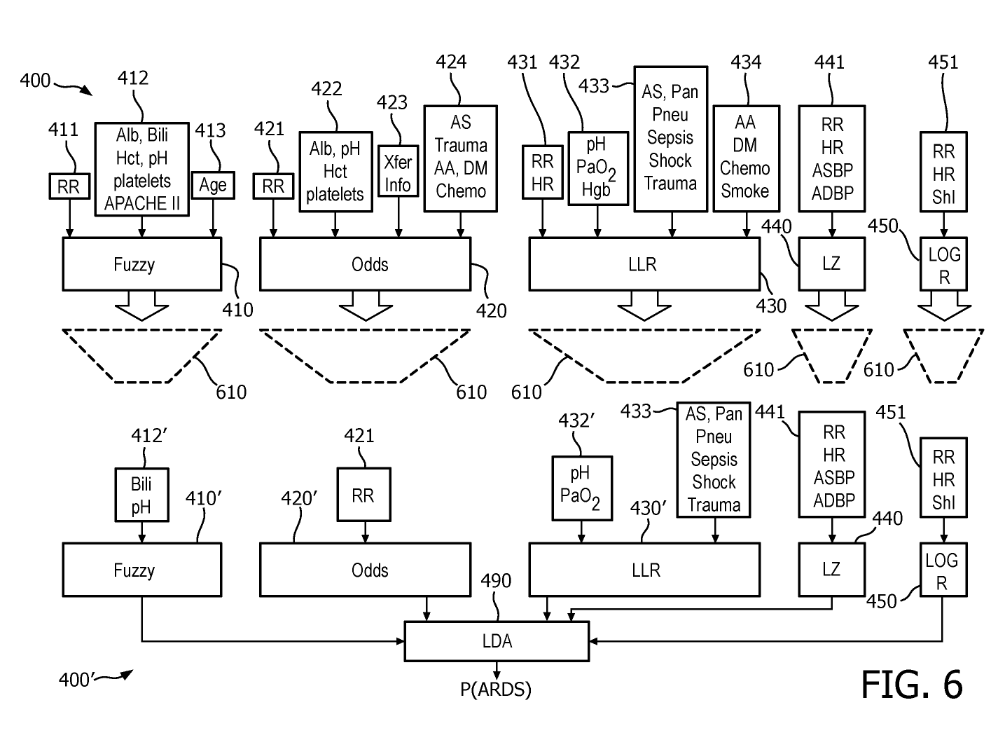

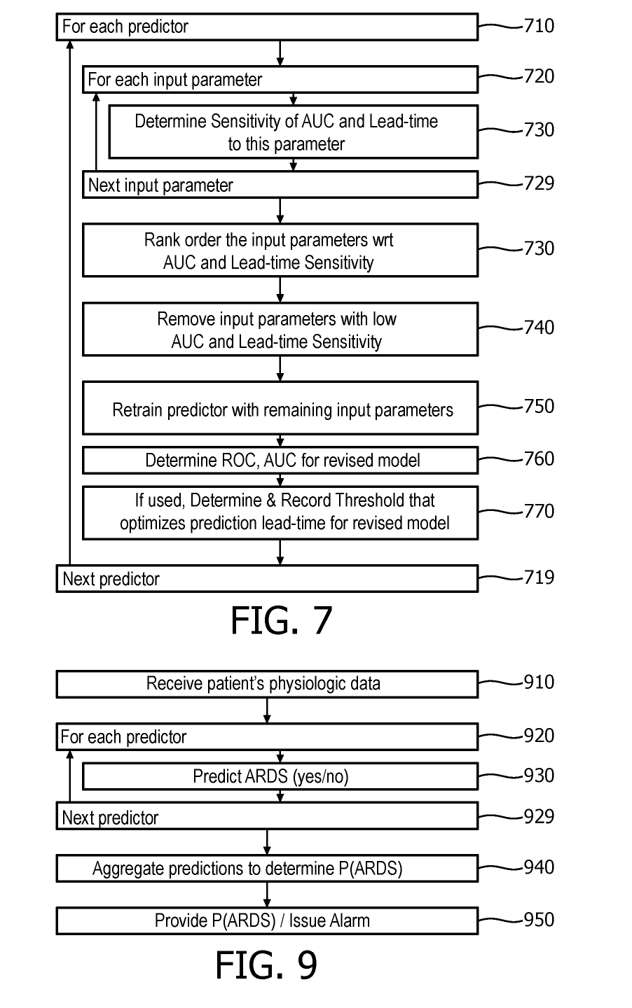

A further reduction in required input data items may be achieved by eliminating data items that do not significantly affect the quality of a diagnostic model. FIG. 6 illustrates an example ‘pruning’ 610 of the ARDS detection system 400 to reduce the number of required input features to each diagnostic model. FIG. 7 illustrates an example flow diagram for pruning of the ARDS detection system 400, such as might be used in the pruning block 610. The term ‘predictor’ is used in FIG. 7, because, as detailed further below, the inputs to the aggregator may also be assessed to potentially eliminate the output of one or more of the diagnostic models.

The loop 710-719 assesses each diagnostic model independently for each input feature. One of skill in the art may recognize that a multi-variate and/or interdependent assessment may be performed. For example, if it is determined that a particular input feature has a significant impact on a particular diagnostic model and cannot be eliminated from that diagnostic model, that input feature may be omitted from consideration by other diagnostic models because its elimination from the other diagnostic models will not reduce the overall number of inputs required by the diagnostic system 400.

Within the loop 720-729, the sensitivity of the diagnostic model to each input feature is assessed, at 730, using a plurality of physiological and observable measures of prior patients for whom the actual onset or non-onset of ARDS is known.

Techniques for determining a process's sensitivity to values of an input feature are well known in the art, and may generally be characterized as statistical techniques or empirical techniques. Statistical tests include, for example, Analysis of Variance (ANOVA) wherein the contribution of each input feature to the variance of an output of interest is determined. An input feature that significantly contributes to the variance of the output of interest can be expected to significantly affect the value of the output of interest.

Empirical techniques may include, for example, ‘what-if’ analyses: ‘what if’ the input feature had a minimum value: how would the output of interest change?; ‘what if’ the input feature had a maximum value: how would the output of interest change?; and so on.

If the diagnostic model allows an input variable to be omitted without changing the internals of the diagnostic model, an empirical assessment may include merely reprocessing the prior patient data without the input variable and observing how the output of interest varies.

In the example pruning element 610, there are two outputs of interest: the AUC of the diagnostic model and the prediction lead time. The sensitivities of each of the AUC and prediction lead time to each input feature are compared to rank-order the input features with regard to each of these outputs of interest, at 730. The prediction lead time may be measured by the mean or median of the time of prediction before the onset, or it may be measured by the proportion of predictions before a given minimum prediction lead time, and so on. If the AUC and prediction lead time are both relatively insensitive to the value of an input feature, then that input feature may be eliminated from the current detector, at 740.

Different criteria may be used to define relative insensitivity for each of the AUC and the prediction lead time. A change of less than 5% in the AUC may be considered to indicate a relative insensitivity of the AUC to the input feature, for example; but, because prediction lead time may be crucial to the patient's recovery, a change of less than 1% in the prediction lead time may be required before the input feature is considered to have an insignificant effect on the prediction lead time. In an example embodiment, each of the input features may be rank ordered based on the sensitivity of the diagnostic model to each input feature and the “top N” input features providing the highest sensitivity may be selected for use in the revised diagnostic model. In an alternative embodiment, a weighted ranking may be used based on the relative cost or degree of invasiveness of obtaining each input feature. That is, if it is relatively easy to obtain a particular input feature, the criteria for retaining that feature may be lower than the criteria for retaining an input feature that is difficult to obtain.

After the input features that provide low sensitivity are eliminated from a diagnostic model, the diagnostic model may be retrained to optimize its performance using the reduced set of input features, at 750, and the ROC and AUC of this revised diagnostic model may be determined, at 760. Of particular note, at 770, if the diagnostic model's binary output after thresholding is used as the output of the diagnostic model, for subsequent aggregation, or for issuing an alert from the aggregator, the revised diagnostic model is assessed to identify a threshold value that optimizes the prediction lead time, using, for example, the process detailed above with regard to FIG. 5.

After all of the diagnostic models are trained and, for those diagnostic models that provide a binary output after thresholding, provided with a threshold that optimizes the prediction lead time, the process terminates, at 719.

As illustrated in FIG. 6, a revised fuzzy inference diagnostic model 410′ that requires only the Bili and pH input features replaces the original diagnostic model 410 that had required all of the inputs 411-413. A revised odds ratio diagnostic model 420′ that requires only respiration rate (RR) replaces the original diagnostic model 420 that had required all of the inputs 421-424. A revised log likelihood ratio diagnostic model 430′ requiring Ph, PaO2, AS, Pan, Pneu, Sepsis, Shock, and Trauma input features replaces the original diagnostic model 430 that had required all of the inputs 431-434.

The Lempel-Ziv diagnostic model 440 and the Logistic regression diagnostic model 450 were found to require all of the original inputs 441, 451, respectively, and remained unchanged. That is, either the AUC or the prediction lead time, or both, of the diagnostic models 440, 450 were found to be sensitive to each of the input features 441, 451, respectively.

As can be seen, the parsimonious ARDS detection system 400′ requires substantially fewer inputs than the ARDS detection system 400, with minimal, if any, effect on the quality of the prediction (AUC) or the prediction lead time, as detailed further below.

As noted above, further reduction of input requirements may be achieved by subjecting the aggregator 490 to the pruning process of FIG. 7. That is, the sensitivity of the AUC and prediction lead time provided by the aggregator 490 to each of the inputs to the aggregator 490 may be determined, and the inputs having an insignificant effect on the AUC and prediction lead times may be eliminated. One will recognize that a consequence/benefit of removing an input to the aggregator 490 is the elimination of the diagnostic model that provided that input, and all of its input features.

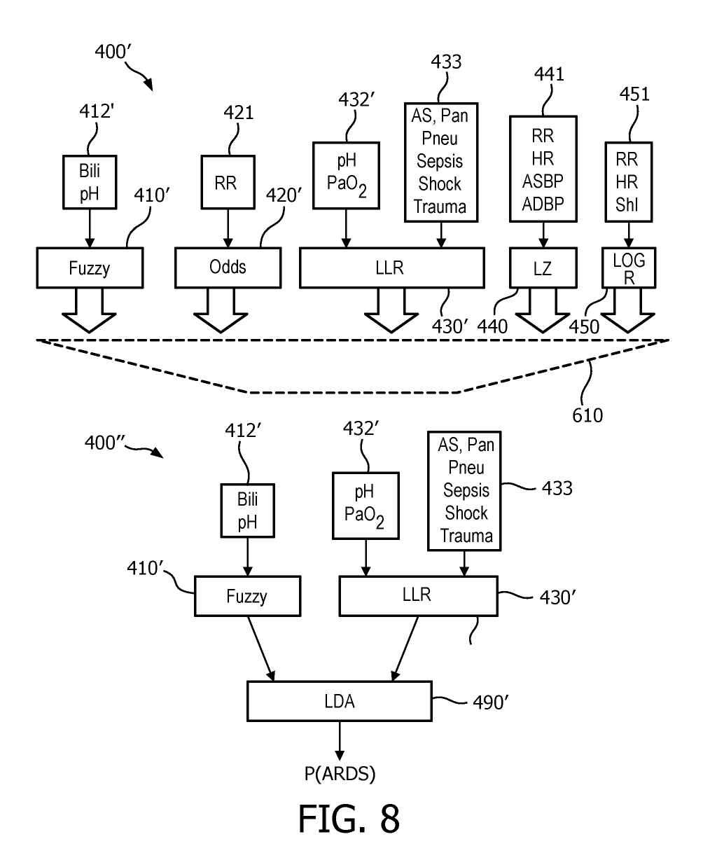

FIG. 8 illustrates a further example pruning of the ARDS detection system of FIG. 6. Using the physiological and observable measures of prior patients, and their actual ARDS outcome, it was determined that the input from the odds likelihood diagnostic model 420′, the Lempel-Ziv diagnostic model 440, and the logistic regression diagnostic model 450 had an insignificant effect on either the AUC or the prediction lead time of the aggregator 490, and these diagnostic models were eliminated as inputs to the revised aggregator 490′ of the ARDS detection system 400″.

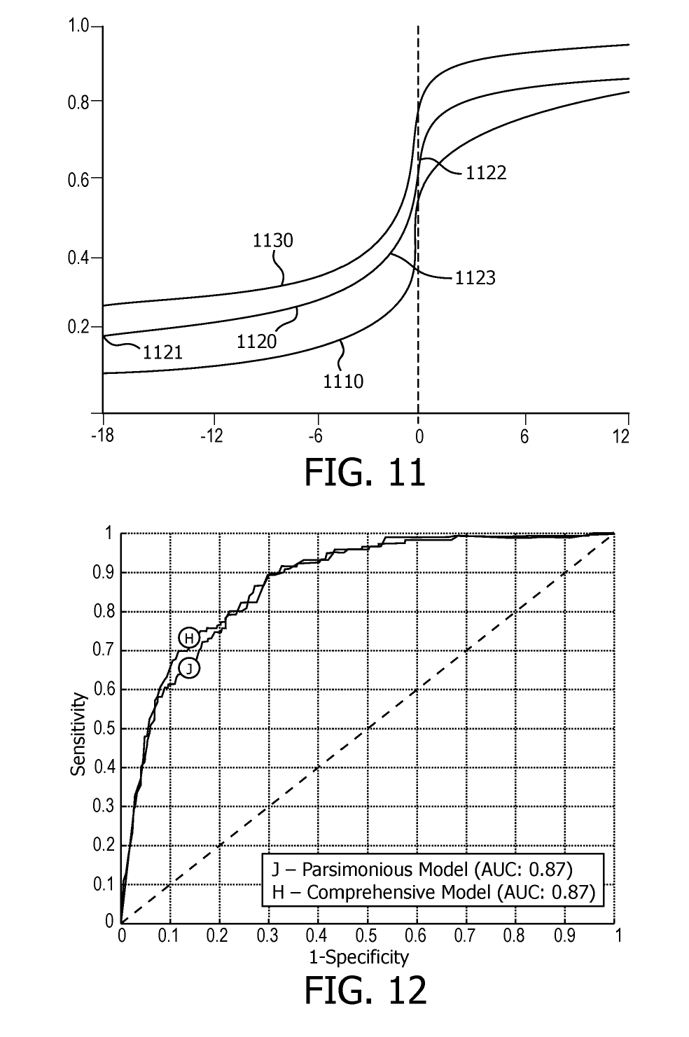

FIG. 12 illustrates example ROC curves H and J corresponding to the ROC H of the prior art comprehensive model of FIG. 3 (curve H in FIG. 4), and the ROC J of the parsimonious model of FIG. 8. As can be seen, the parsimonious model provided by this invention offers comparable prediction performance as the prior art comprehensive model.

FIG. 9 illustrates an example flow diagram for using an embodiment of this invention for detecting an onset of ARDS.

At 910, the patient's physiological data is received. This data is generally the most recent data of the patient, but if a given diagnostic model uses comparative values, such as a change of value of a data element, time series data for the patient may also be provided. This data, or a subset of this data, is provided to each diagnostic model to obtain a prediction of whether or not the patient is likely to experience ARDS in the loop 920-929.

At 930, the data is assessed to determine whether the available patient data is sufficient to provide the data required by the diagnostic model. If the data is not available for this patient, an artificial or predicted value is provided, at 930. This artificial value may be obtained based on data values of prior patients with similar characteristics as the current patient, prior data values of the current patient, average values of the population at large, or other sources. A confidence interval may be associated with this artificial value based on the estimated variance associated with this value. The variance may be based on the distribution of data values among the population from which the artificial value is selected, based on a variance provided by a medical reference with regard to the particular physiological element, based on a known feasible range of the data values, or other techniques.

At 940, the diagnostic model receives the patient data and artificial data, if any, and produces an ARDS prediction. This prediction may be a binary (yes/no) prediction based on whether a computed measure based on the input data is above or below a given threshold for this diagnostic model. The given threshold may have been selected to maximize the proportion of true positive predictions while allowing for a given proportion of false positive predictions. Optionally, the prediction may be a numeric value that is subsequently consolidated with other numeric values and compared to a consolidated threshold to provide an aggregated binary prediction.

After all of the diagnostic models provide a prediction based on the patient data, an aggregate prediction is provided, at 950. One of skill in the art will recognize that if the aggregator is considered to be a predictor in the loop 920-929, this step 950 may merely correspond to steps 930-940 being applied to this ‘last’ predictor.

The aggregate prediction is then output from the system as a calculated probability that the patient is likely to experience ARDS, and/or as an alarm notification if the predicted output is greater than the selected threshold, i.e. if the prediction of ARDS is positive.

If any artificial data was used as input to a diagnostic model, the output of the diagnostic model may be a plurality of predictions, based on the variance associated with the artificial data. For example, if the variance is the conventional computed variance statistic, the diagnostic model may be provided a first input equal to the artificial value plus twice the variance, and a subsequent second input equal to the artificial value minus twice the variance (the “two sigma” values) to provide two corresponding predictions. In other cases, the inputs to the diagnostic model may be the extents of the known feasible range of the data values. In other cases, the inputs to the diagnostic model may be the artificial value plus or minus a given percentage of the artificial value. One of skill in the art will recognize that other input values representative of a range of values that the data item may assume may also be used.

Assuming that the different variance-dependent input values provide a plurality of different predictions, this plurality is provided to the aggregator that combines the predictions, and the aggregator processes each of the plurality of predictions to determine the predicted output assuming that the data item might have produced each of these predictions. If the output of the aggregator differs depending upon the different variance-dependent outputs of the individual diagnostic model, both outputs may be presented with an identification of which input was missing, causing the conflicting outputs. The user of the system is thus advised of which input will serve to remove the ambiguity in the prediction.

If the different variance-dependent input values all produce the same prediction, that single prediction is provided as the output of the predictor, with no variance associated with the prediction. This applies to the individual diagnostic models as well as the aggregator.

While the invention has been illustrated and described in detail in the drawings and foregoing description, such illustration and description are to be considered illustrative or exemplary and not restrictive; the invention is not limited to the disclosed embodiments. For example, in the aforementioned pruning processes, a multivariate pruning may be performed, wherein the sensitivity of each diagnostic model is assessed for a combination of input features. That is, it may be found that the sensitivity of the model on a pair of input features is substantially greater than any individual sensitivity, which may enable the elimination of other input features provided that this pair of features is not eliminated.

Additionally, it is possible to operate the invention in an embodiment wherein the steps described could be used to optimize a different set of diagnostic models for detecting a different clinical event such as Acute Kidney Injury or Acute Hypotension.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A non-transitory computer readable medium that includes a program that, when executed by a processor, causes the processor to:
receive a plurality of diagnostic models for predicting Acute Respiratory Distress Syndrome (ARDS), each diagnostic model being configured to receive a corresponding set of input features and to produce therefrom a prediction of an onset of ARDS;
for each of the diagnostic models:
provide a time series of physiological data of each patient of a plurality of prior patients, an identification of whether the patient experienced ARDS, and a time of ARDS onset for each patient that experienced ARDS; the physiological data corresponding to each of the input features to the diagnostic model;
determine a Receiver Operating Characteristic (ROC) curve and an area under the ROC curve (AUROC) that characterizes the diagnostic model's ability to correctly identify whether a patient will experience ARDS or not;
for each input feature of the diagnostic model: determine a rank order of the input feature based on at least the input feature's impact on the ROC curve;
select a subset of the input features based on the rank order of the input features; and
if the subset includes fewer than a total number of the input features of the diagnostic model:
create a revised diagnostic model that uses only the subset of the input features; and
store the revised diagnostic model as the diagnostic model to be subsequently used to predict an onset of ARDS by an other patient;
wherein the subset of at least one diagnostic model includes fewer than a total number of the input features of the diagnostic model.

2. The medium of claim 1, wherein the program causes the processor to select a threshold for an aggregation of the predictions of the diagnostic models that maximizes an early detection of ARDS while providing not more than a predefined acceptable proportion of false positive predictions; and wherein the rank order of the input features is also based on a time of early detection using the selected threshold.

3. The medium of claim 2, wherein the program causes the processor to determine a threshold for at least one diagnostic model that maximizes an early detection of ARDS while providing not more than an acceptable proportion of false positive predictions.

4. The medium of claim 3, wherein the program causes the processor to determine a threshold for each of the diagnostic models that maximizes an early detection of ARDS while providing not more than an acceptable proportion of false positive predictions, and the aggregation of the predictions is based on a binary (ARDS, not-ARDS) output of each of the diagnostic models based on the threshold of each diagnostic model.

5. The medium of claim 4, wherein the aggregation of the predictions is based on a SOFALI voting system.

6. The medium of claim 2, wherein one or more of the diagnostic models provide a non-binary value of the prediction, and the aggregation of the predictions is based on a Linear Discrimination Analysis (LDA).

7. The medium of claim 1, wherein the program causes the processor to:
receive a set of physiological data of the other patient;
provide the set of physiological data of the other patient to each of the plurality of diagnostic models to determine a plurality of predictions of ARDS;
combine the plurality of predictions to provide a composite likelihood of ARDS;
compare the composite likelihood to the selected threshold to determine a binary (positive/negative) prediction of ARDS, and
report the binary prediction of ARDS for this other patient.

8. The medium of claim 7, wherein the program causes the processor, upon determining that a value of an element of the set of physiological data of the other patient is missing for one or more of the diagnostic models, to:
provide an artificial value for the missing value; and
determine a range of the artificial value based on a variance associated with the artificial value;
wherein:
providing the set of physiological data to the one or more revised diagnostic models includes providing a plurality of values within the range of the artificial value to the one or more revised diagnostic models to determine a confidence interval about the prediction of ARDS based on providing the artificial value for the missing value;
combining the plurality of predictions includes determining the composite likelihood of ARDS includes assessing the likelihood of ARDS with respect to the confidence interval about each prediction.

9. The medium of claim 1, wherein the plurality of diagnostic models include two or more of:
a fuzzy logic model;
an odds ratio model;
a log-likelihood model;
a Lempel-Ziv complexity model; and
a logistic regression model.

10. A medical diagnostic system comprising:
a plurality of diagnostic models that are each configured to provide a prediction of a patient's risk of experiencing Acute Respiratory Distress Syndrome (ARDS), based only on the patient's physiological data; and
an aggregator that is configured to aggregate the predictions of the plurality of diagnostic models to provide an aggregated prediction of an onset of ARDS based on the patient's physiological data;
wherein at least the aggregator is configured to provide a binary (positive/negative) prediction based on a select threshold value, and
the select threshold value is selected to provide a maximum proportion of early detections of ARDS while providing a maximum allowable proportion of false positive predictions.

11. The medical diagnostic system of claim 10, wherein the maximum allowable proportion of false positives is at least 25%.

12. The medical diagnostic system of claim 10, wherein, if the patient's physiological data is insufficient for providing a required input to at least one diagnostic model, the system provides an artificial value for the required input, and a variance associated with the artificial value, and the at least one diagnostic model is configured to provide a confidence interval about its prediction based on the variance associated with the artificial value.

13. The medical diagnostic system of claim 10, wherein the aggregator includes a Linear Discrimination Analysis (LDA) system.

14. The medical diagnostic system of claim 10, wherein the prediction of each diagnostic model includes a binary (ARDS, not-ARDS) prediction, and the aggregator includes a voting system.

15. The medical diagnostic system of claim 14, wherein the binary prediction of each diagnostic model is based on a threshold value of the model that is selected to provide a maximum proportion of early detections of ARDS while providing less than a maximum allowable proportion of false positive predictions.
